(19) United States
(12) Patent Application Publication
(10) Pub. No.: US 2012/0302917 A1
(43) Pub. Date: Nov. 29, 2012

(54) Urine Meter

(75) Inventors: Alan Fitzgerald, Edgeworthstown (IE); Ciara Deighan, Birr (IE); Paul J. Daly, Tullamore (IE); Tom Robinson, Roscrea (IE)

(73) Assignee: TYCO HEALTHCARE GROUP LP, Mansfield, MA (US)

(21) Appl. No.: 13/477,309

(22) Filed: May 22, 2012

(30) Foreign Application Priority Data
May 23, 2011 (EP) 11004231.4

(51) Int. Cl. A61B 5/20

(52) U.S. Cl. 600/575

(57) ABSTRACT

The present disclosure provides a urine meter (40) having a housing (14) which includes an inlet (16) and an outlet (20). The housing (14) further includes a first measurement chamber (24) and a second measurement chamber (26) arranged such that liquid entering the inlet (16) enters the first measurement chamber (24) before passing to the second measurement chamber (26). The housing (14) is further provided with a non-return valve (42) between the first and second measurement chambers (24,26) which, in use, prevents the flow of liquid from the second measurement chamber (26) to the first measurement chamber (24).

### BACKGROUND

1. Technical Field

The present invention relates to a urine meter, and in particular to a urine meter for monitoring the flow of urine from a catheterised patient.

2. Background of Related Art

FIG. 1 shows a known urine meter, generally designated 10, and a urine collection bag 12. The urine meter 10 includes a housing 14 that is provided with an inlet 16 having an inlet conduit 18. The inlet conduit is in use, connectable to a urinary catheter of a patient. The housing 14 is further provided with an outlet 20 which is connected by an outlet conduit 22 to the collection bag 12. Straps 30 are provided on the urine meter 10 to permit both the meter 10 and collection bag 12 to be mounted to an appropriate support. The support may, for example, be the rail of a patient bed.

The housing 14 further defines first, second and third measurement chambers 24, 26, 28. For the urine meter 10 shown, the first measurement chamber 24 has a volume of 100 millilitres, the second measurement chamber 26 has a volume of 200 millilitres, and the third measurement chamber 28 has a volume of 200 millilitres. The housing 14 is formed from a clear plastic material such as, for example, a styrene-butadiene copolymer. One such suitable material is K-Resin® SBC. The interior of each measurement chamber 24, 26, 28 is viewable through the housing 14, and each chamber 24, 26, 28 is marked with graduations which permit the volume of liquid in each chamber 24, 26, 28 to be measured. The first chamber 24 is marked with graduations from 0 ml to 100 ml, the second chamber 26 is marked with graduations from 100 ml to 300 ml, and the third chamber is marked with graduations from 300 ml to 500 ml. The first chamber 24 is separated from the second chamber 26 by an interior wall 32 of the housing 14 which defines a weir between the chambers 24, 26. The second chamber 26 is similarly separated from the third chamber 28 by a further interior wall 34 of the housing 14 which defines a weir between the chambers 26, 28.

The housing 14 is configured such that liquid entering the meter 10 through the inlet 16 flows firstly into the first chamber 24. Once the first chamber 24 has been filled, incoming liquid spills over the weir defined by the wall 32 separating the first and second chambers 24, 26 and into the second chamber 26. Once the second chamber 26 has been filled, incoming liquid spills over the weir defined by the wall 34 separating the second and third chambers 26, 28 and into the third chamber 28. The urine meter 10 is further provided with an overflow conduit 36 which connects the third chamber 28 to the outlet 20. In the event that the first, second and third chambers 24, 26, 28 become filled, excess liquid is conducted through the overflow conduit 36 to the outlet 20.

The meter 10 is further provided with a drainage tap 38 which is located in a lower region of the housing 14. The drainage tap 38 enables the chambers 24, 26, 28 to be placed in fluid communication with the outlet 20 and thereby permit liquid present in the chambers 24, 26, 28 to be drained to the collection bag 12. The drainage tap 38 is configured so as to permit the sequential drainage of the chambers 24, 26, 28.

One drawback of the urine meter 10 of FIG. 1 is that in the event of the meter 10 being tipped or shaken, for example when the meter 10 is moved, liquid can transfer between the chambers 24, 26, 28 by passing over the chamber separating walls 32, 34. This can thus lead to the meter 10 providing an incorrect reading.

### SUMMARY

According to the present invention there is provided a urine meter having a housing which includes an inlet and an outlet, the housing further including a first measurement chamber and a second measurement chamber arranged such that liquid entering the inlet enters the first measurement chamber before passing to the second measurement chamber, wherein the housing is further provided with a non-return valve between the first and second measurement chambers which, in use, prevents the flow of liquid from the second measurement chamber to the first measurement chamber.

The non return valve may be biased to an open condition to permit fluid communication between the first chamber and the second chamber, and urged to a closed condition to prevent fluid communication between the chambers by the action of liquid present within the second chamber. In such an embodiment the non return valve may be biased to the open condition by gravity. The non return valve may be urged to the closed condition by the impact of liquid contained in second chamber. Such impact may occur as a result of sloshing of the liquid within the second chamber resulting from movement of the meter. The non return valve may be urged to the closed condition due to the buoyancy of a portion of the non return valve. In such an embodiment, the non return valve is urged to the closed condition by the rising free surface of the liquid.

The non return valve is provided in an internal partition of the housing which separates the first chamber from the second chamber. The non return valve may include a valve member which is mounted to the internal partition, and at least one aperture which extends through the internal partition and which defines fluid communication channel between the first and second measurement chambers. In such an embodiment the valve member may include a stem and a head, wherein the head defines a sealing surface which is movable into contact with the internal partition to close the at least one aperture. The sealing surface many be an annular sealing surface.

The stem of the valve member may received in an aperture of the internal partition. In such an embodiment, the valve member may be retained in the aperture of the internal partition by the head of the valve member and a formation on the stem of the valve member. The formation on the stem of the valve member is resiliently deformable so as to enable the valve member to be fitted to the aperture of the internal partition. The internal partition is provided with a plurality of apertures which extend through the internal partition and which define fluid communication channels between the first and second measurement chambers.

In an alternative embodiment, the housing including a third measurement chamber which, in use receives liquid from the second measurement chamber, wherein the housing is provided with a further non-return valve between the second and third measurement chambers which, in use, prevents the flow of liquid from the third measurement chamber to the second measurement chamber. In such an embodiment it will be appreciated that the non return valve may have the same configuration and operating characteristics as non return valve described with reference to the previously described two chamber embodiment.

In such an embodiment the further non-return valve is preferably positioned lower in the housing than the first non-return valve. This ensures liquid is able to flow from the second measurement chamber to the third measurement chamber before closure of the non-return valve provided between the first and second measurement chambers.

### BRIEF DESCRIPTION OF THE DRAWINGS

An embodiment of the present invention will now be described with reference to the accompanying drawings in which:

FIG. 1 shows a known urine meter and collection bag;

FIG. 2 shows a cross-sectional view of a urine meter according to the present invention;

FIG. 3 shows a cross-sectional view of a non-return valve of the urine meter of FIG. 2, the valve being in an open position;

FIG. 4 shows a cross-sectional view of the non-return valve of the urine meter of FIG. 2, the non-return valve being in a closed position;

FIG. 5 shows a perspective view of the non-return valve;

FIG. 6 shows a perspective view of the valve member; and

FIG. 7 shows a perspective view of the non-return valve with the valve member removed.

### DETAILED DESCRIPTION OF THE EMBODIMENTS

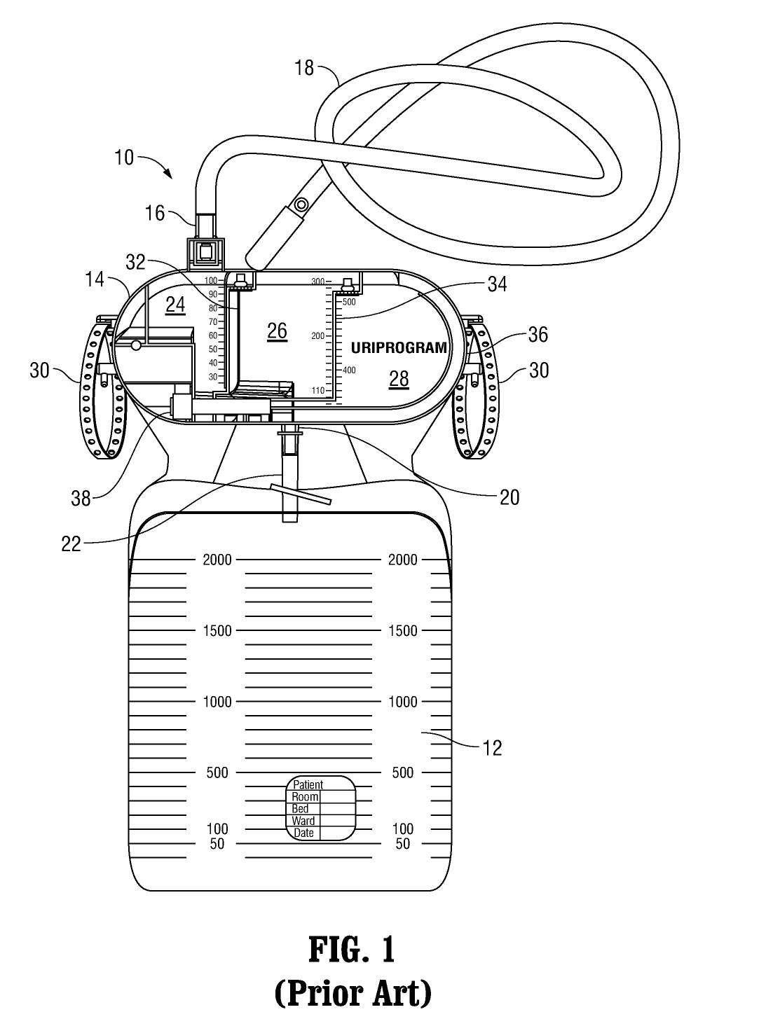

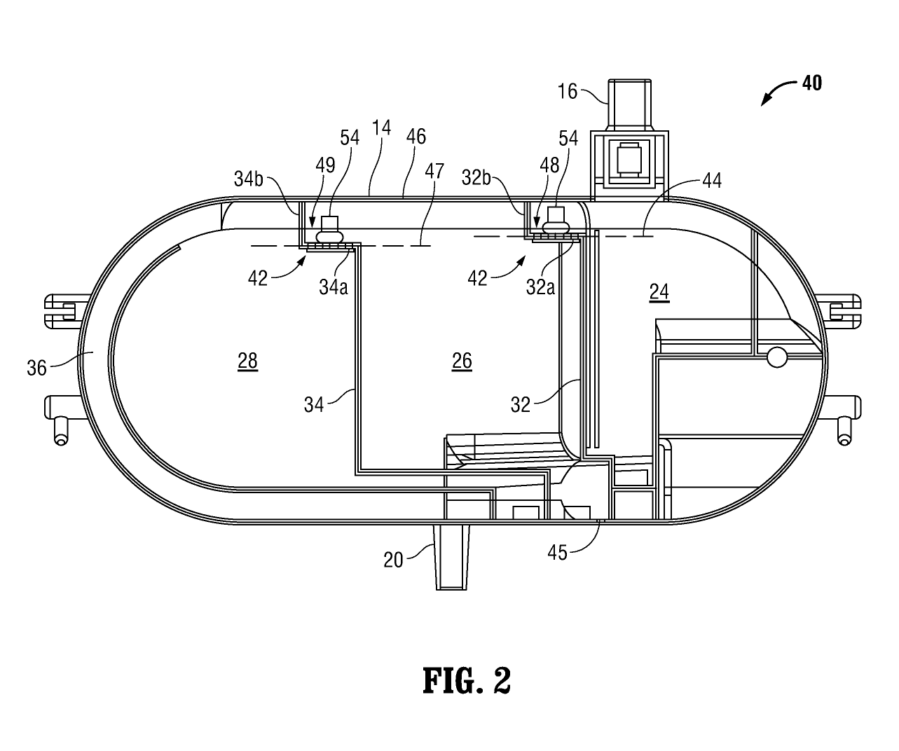

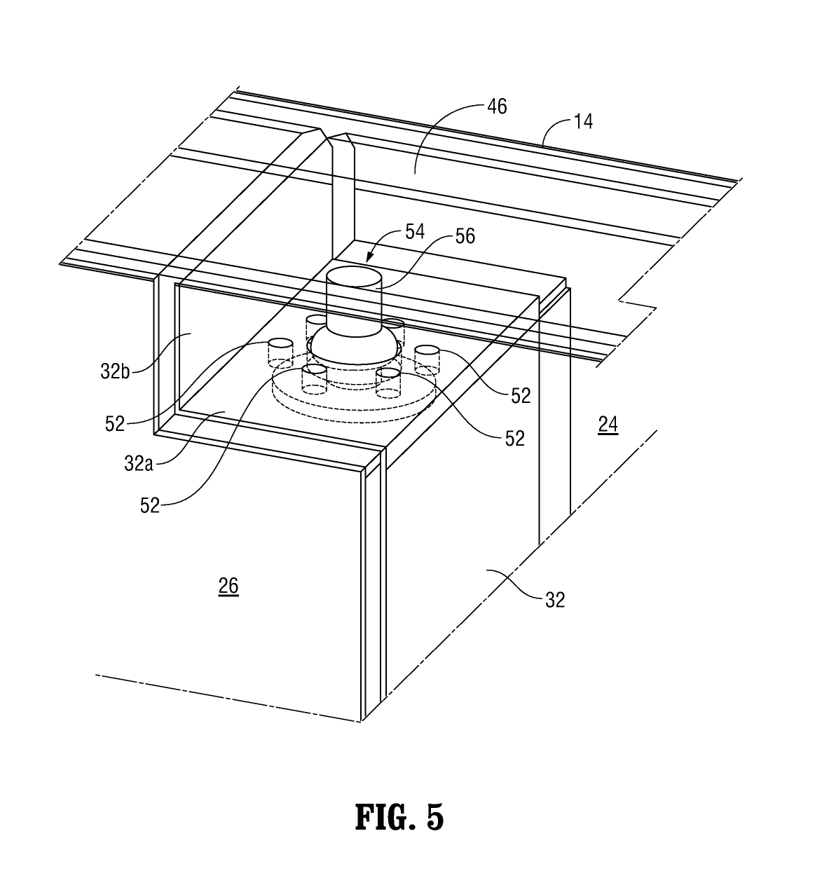

Referring now to FIGS. 2 to 7 there is shown a urine meter, generally designated 40. According to the present invention. Features common to the urine meter 10 described with reference to FIG. 1 are identified with like reference numerals.

FIG. 2 shows a cross-sectional view of the housing 14 of the urine meter 40 viewed from the opposite side to that shown in FIG. 1. The housing 14 of the meter 40 differs from that described with reference to FIG. 1 in that non return valves, generally designated 42, are provided between the first and second chambers 24, 26, and the second and third chambers 26, 28 respectively. Each non return valve 42 is provided in an extension of the wall 32, 34 that separates the first and second chambers 24, 26, and the second and third chambers 26, 28 respectively.

Looking firstly at the wall 32 which separates the first and second chambers 24, 26, the upper edge of the wall 32 in the meter 10 of FIG. 1 is indicated by broken line 44. In the meter 40 of the present invention, the wall 32 is extended such that it extends fully across the housing 14 from the base wall 45 to the top wall 46 thereof. The wall 32 is extended by an extension generally designated 48. The extension 48 includes a first portion 32a which lies on a plane substantially perpendicular to the plane of the major portion of the wall 32. The wall 32 is further extended by a second portion 32b which lies on a plane substantially perpendicular to that of the first portion 32a, and substantially parallel to the plane of the major portion of the wall 32. The first and second portions 32a, 32b of the wall extension 48 thus define a step in the wall 32.

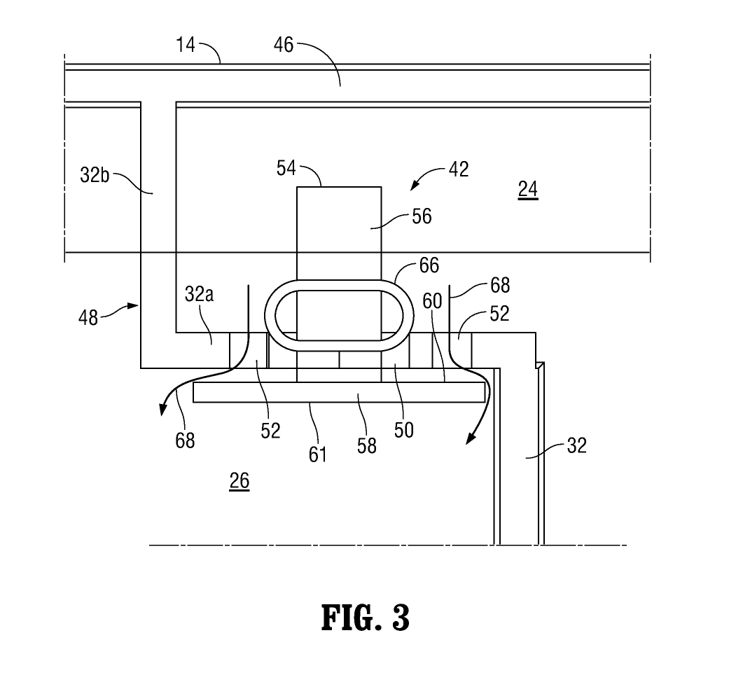

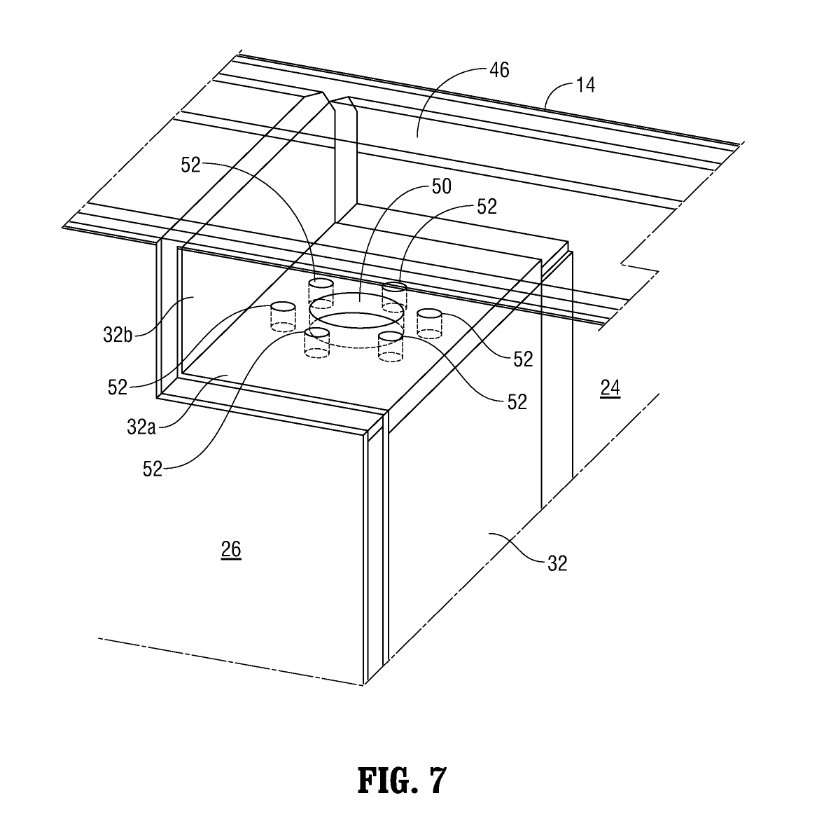

Referring also to FIGS. 3-7, the first portion 32a of the wall extension 48 is provided with an aperture 50 configured to receive a non return valve 42. Aperture 50 may have any cross sectional area which corresponds to a cross sectional area of a portion of the return valve received within aperture 50. Wall extension 48 further comprises at least one second aperture 52 positioned in relation to aperture 50 so that when non return valve 42 is in the open position, fluid may flow through the at least one second aperture 52, and when non return valve 42 is in the closed position, fluid is prevented from flowing through the at least one second aperture 52. The at least one second aperture 52 may, but need not have a cross sectional area less than the cross sectional area of aperture 50. In one embodiment of the invention, the wall extension 48 is provided with a substantially circular first aperture 50 and a plurality of substantially circular second apertures 52 (FIG. 7). In the embodiment shown, the first aperture 50 is provided a substantially central position on the first portion 32a of the wall extension 48. In the embodiment shown, the wall extension 48 is further provided with six second apertures 52 which are equidistantly spaced in a circular arrangement around the first aperture 50. The second apertures 52 have a diameter that is less than that of the first aperture 50. It will be appreciated that the extension 48 may be provided with a greater or lesser number of second apertures 52. As will be described in greater detail below, the first aperture 50 serves to locate and retain a valve member 54 of a non return valve 42, while the second apertures 52, in conjunction with the valve member 54, define fluid conduits extending through the wall 32.

A similar wall extension 49 is provided in the wall 34 that separates the second and third chambers 26, 28. This wall extension 49 similarly includes a first portion 34a which lies on a plane substantially perpendicular to the plane of the major portion of the wall 34. The wall 34 is further extended by a second portion 34b which lies on a plane substantially perpendicular to that of the first portion 34a, and substantially parallel to the plane of the major portion of the wall 34. The wall extension 49 is provided with a similar array of apertures having a first aperture surrounded by a plurality of smaller second apertures. The upper edge of the wall 34 that separates the second and third chambers is indicated by broken line 47 (FIG. 2). The upper edge of the wall 34 is spaced a greater distance from the top wall 46 of the housing 14 than the upper edge of the wall 32 separating the first and second chambers 24, 26. This results in the non-return valve 42 provided between the first and second chambers 24, 26 being positioned higher in the housing 14 than the non-return valve 42 provided between the second and third chambers 26, 28.

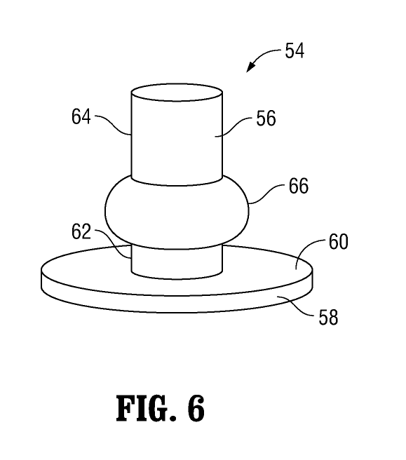

FIG. 6 shows a valve member, generally designated 54, of a non return valve 42. The valve member 54 includes a stem 56 and a head 58. The head 58 of the valve member 54 may have any size and shape suitable to cover all second apertures when the non return valve 42 is in the closed position. In one embodiment, the head 58 of the valve member 54 is substantially disc shaped and is provided with an annular and substantially flat sealing surface 60. Alternately, other valve member configurations are envisioned. The stem 56 extends from a substantially central position on the head 58 such that the annular sealing surface 60 extends around the location at which the stem 56 meets the head 58. The opposite side of the head 58 is provided with a substantially circular face 61. The stem 56 has a first generally cylindrical portion 62, a second generally cylindrical portion 64 and a bulbous portion 66 provided between the first and second generally cylindrical portions 62, 64. The diameter of the first cylindrical portion 62 is substantially equal to the diameter of the second cylindrical portion 64. The diameter of the first and second cylindrical portions 62, 64 is less than the diameter of the first aperture 50 of a wall extension 48 of the housing 14. The bulbous portion 66 has an at rest diameter at its largest cross sectional area that is greater than that of the cylindrical portions 62, 64 and the first aperture 50. The bulbous portion 66 is resiliently deformable so that its diameter can be reduced by compression or elongation. The valve member 54 is manufactured from a flexible plastics material.

FIGS. 3, 4 and 6 show the valve member 54 fitted to the extension 48 of a wall 32 of the housing 14. The valve member 54 is fitted to the extension 48 by first inserting the second cylindrical portion 64 of the stem 56 into the first aperture 50 of the extension 48 from the side of the extension 48 that faces the downstream chamber 26 of the meter 40. The stem 56 is inserted further into the aperture 50 such that the bulbous portion 66 deforms and passes through the aperture 50. Once clear of the aperture 50 the bulbous portion 66 returns to its original shape. It will thus be appreciated that the valve member 54 is thus retained in the aperture 50 by the presence of the bulbous portion 66 of the stem 56 on one side of the aperture 50, and the head 58 of the valve member 54 on the other side of the aperture 50. The outer diameter of the bulbous portion 66 which contacts a first surface of wall extension 48 is smaller than an inner cross sectional area of a periphery defined by each innermost point of each of the at least one second aperture 52 so that the bulbous portion does not interfere with flow through the at least one second aperture 52. The cross sectional area of the sealing surface 60 is greater than an outer cross sectional area of a periphery defined by each outermost point of each of the at least one second aperture 52 so that the sealing surface 60 interrupts flow through the at least one second aperture 52 when the sealing surface 60 contacts a second surface of wall extension 48. In one embodiment, the outer diameter of the sealing surface 60 of the head 58 is greater that the outer diameter of the circular arrangement of the second apertures 52.

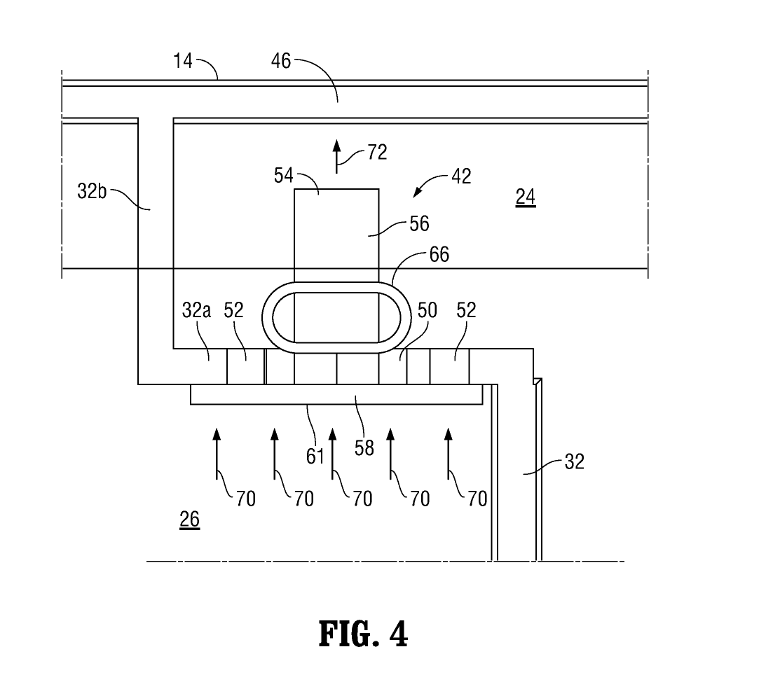

The length of the first cylindrical portion 62 of the stem 56 is greater than the thickness of the portion 32a of the wall extension 48 in which the first aperture 50 is provided. It will thus be understood that the valve member 54 is able to move between an open position, shown in FIG. 3, where the bulbous member 66 abuts the first aperture and the sealing surface 60 is spaced from the wall extension 48, and a closed position, shown in FIG. 4, where the sealing surface 60 abuts the wall extension 40 and the bulbous member 66 is spaced from the first aperture 50.

In use, the valve member 54 is gravity biased towards the open position shown in FIG. 3. In the first position, the arrangement of second apertures 52 are open and each second aperture 52 defines a fluid communication path through the wall extension 48. Liquid is this able to pass from the upstream chamber 24 to the downstream chamber 26 as indicated by arrows 68. In the event that liquid attempts to flow back from the downstream chamber 26 to the to the upstream chamber the liquid contacts the circular face 61 of the head 58 as indicated by arrows 70 and urges the valve member 54 to the closed position as indicted by arrow 72. The sealing surface 60 abuts a second surface of the wall extension 48 and thus closes the second apertures 52. Liquid may come into contact with the circular face 61 as a result of the downstream chamber 26 filling to the level of the first portion 32a of the wall extension 48. In such an instance, the valve member 54 is moved to the closed position due to buoyancy of the valve member 54 in the liquid. Alternatively, in instances where the downstream chamber 26 is only partially filled, the valve member 54 may be urged to the closed position as a result of sloshing of the liquid contained in the downstream chamber 26.

As described above, the non return valve 42 provided between the first and second chambers 24, 26 is positioned at a higher level within the housing 14 than the non return valve 42 provided between the second and third chambers 26, 28. During normal use of the urine meter 40, i.e. when it is positioned level and stably mounted to, for example, a bed frame, the differing non- return valve heights permit the chambers 24, 26, 28 to fill sequentially. The upper edge of the wall 34 separating the second and third chambers 26, 28 is provided below the level adopted by the circular face 61 of the non return valve 42 between the first and second chambers 24, 26 in the open position. As such, it will be appreciated that upon filling of the second chamber 26 to the level of the top of the wall 34 separating the second and third chambers 26, 28, further liquid introduced into the second chamber 26 will flow into the third chamber 28.

In the embodiment of the invention described above, the urine meter 40 is provided with three measurement chambers 24, 26, 28. It will be appreciated that non-return valves 42 of the present invention may be provided in urine meters having two measurement chambers or more than three measurement chambers.

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described embodiments. Accordingly, the disclosure is not to be limited by what has been particularly shown and described, except as indicated by the appended claims.

What is claimed is:

1. A urine meter having a housing which includes an inlet and an outlet, the housing further including a first measurement chamber and a second measurement chamber arranged such that liquid entering the inlet enters the first measurement chamber before passing to the second measurement chamber, wherein the housing is further provided with a first non-return valve between the first and second measurement chambers which, in use, prevents the flow of liquid from the second measurement chamber to the first measurement chamber.

2. A urine meter as claimed in claim 1 wherein the first non return valve is biased to an open condition to permit fluid communication between the first chamber and the second chamber, and is urged to a closed condition to prevent fluid communication between the chambers by the action of liquid present within the second chamber.

3. A urine meter as claimed in claim 2 wherein the first non return valve is biased to the open condition by gravity.

4. A urine meter as claimed in claim 1 wherein the first non return valve is supported on an internal partition of the housing which separates the first chamber from the second chamber.

5. A urine meter as claimed in claim 4, wherein first the non return valve includes a valve member which is mounted to the internal partition, and at least one aperture which extends through the internal partition and which defines at least one fluid communication channel between the first and second measurement chambers.

6. A urine meter as claimed in claim 5 wherein the valve member includes a stem and a head, wherein the head defines a sealing surface which is movable into contact with the internal partition to close the at least one aperture.

7. A urine meter as claimed in claim 6 wherein the sealing surface is an annular sealing surface.

8. A urine meter as claimed in claim 6 wherein the stein of the valve member is received in an aperture of the internal partition.

9. A urine meter as claimed in claim 8 wherein the valve member is retained in the aperture of the internal partition by the head of the valve member and a formation on the stem of the valve member.

10. A urine meter as claimed in claim 9 wherein the formation on the stem of the valve member is resiliently deformable so as to enable the valve member to be fitted to the aperture of the internal partition.

11. A urine meter as claimed in claim 5 wherein the at least one aperture includes a plurality of apertures which extend through the internal partition and the at least one fluid communication channel includes a plurality of fluid communication channels between the first and second measurement chambers.

12. A urine meter as claimed in claim 1, wherein the housing includes a third measurement chamber which, in use, receives liquid from the second measurement chamber, wherein the housing is provided with a second non-return valve between the second and third measurement chambers which, in use, prevents the flow of liquid from the third measurement chamber to the second measurement chamber.

13. A urine meter as claimed in claim 12 wherein the second non-return valve provided between the second and third measurement chambers is positioned lower in the housing than the first non-return valve provided between the first and second measurement chambers.
